(19) United States
(12) Patent Application Publication
(10) Pub. No.: US 2009/0283094 A1
(43) Pub. Date: Nov. 19, 2009

(54) MEDICINE EJECTION DEVICE

(75) Inventors: Soji Hamano, Yokohama-shi (JP); Toshiyuki Nobutani, Yokohama-shi (JP); Keisuke Kawahara, Tokyo (JP); Masaru Sugita, Tokyo (JP)

Correspondence Address: FITZPATRICK CELLA HARPER & SCINTO, 1290 Avenue of the Americas, NEW YORK, NY, 10104-3800, US

(73) Assignee: Canon Kabushiki Kaisha, Tokyo (JP)

(21) Appl. No.: 12/373,905

(22) PCT Filed: Jun. 29, 2007
(86) PCT No.: PCT/JP2007/063514

§ 371 Date: Jan. 15, 2009

(30) Foreign Application Priority Data
Jul. 19, 2006 (JP) 2006-196327

(51) Int. Cl. A61M 15/00

(52) U.S. Cl. 128/203.15

(57) ABSTRACT

In a medicine ejection device for administering a medicine to the throat, bronchi or lungs, the air flow path to which the medicine has stuck is to be kept in clean state in a simple configuration. The medicine ejection device which ejects a medicine to be inhaled by a user via a suction port, including a flow path member linked to the suction port and capable of forming an air flow path in which the medicine is guided by the inhalation by the user to the suction port, and an internal wall face constituting member which is intended for formation of the internal wall of the air flow path and allows detachable fitting in the flow path member.

### TECHNICAL FIELD

The present invention relates to a medicine ejection device for ejecting a medicine to be inhaled by a user.

### BACKGROUND ART

The medicine ejection devices disclosed in International Publication No. WO95/01137 and International Publication No. WO02/04043 are bringing to realization treatments of users who utilize information databases including electronic medical charts in combination. Such medicine ejection devices have a memory unit for storing personal information on users including information on their medical charts and prescriptions. These medicine ejection devices also are portable terminals also serving as inhalers to have medicines inhaled by their users. They also have a spraying control unit which controls inhalers according to each user's inhalation profile and sprays a medicine so that he or she can inhale the medicine in accordance with information from the prescription.

Within such a medicine ejection device, there usually is a passage over which atomized medicine is carried (hereinafter in this specification referred to as an “air flow path”) from the point where the medicine in the device until it is administered into the user's body from the portion ejecting a medicine within the device. In this process, part of the ejected medicine may conceivably stick to the internal wall face of the air flow path. Leaving it intact might invite multiplication of various germs on the internal wall of the air flow path where the medicine has stuck, and would be undesirable from the sanitary point of view. Thus, conventional medicine ejection devices involve an unsolved problem that, because the internal wall of the air flow path in the device is smeared when the patient inhales, the member itself constituting the air flow path should be taken out and washed or replaced with a new member.

An object of the present invention, attempted in view of the unsolved problem noted above, is to provide a medicine ejection device which permits ready remedying of contamination of the air flow path in the medicine ejection device.

### DISCLOSURE OF THE INVENTION

In view of the problem noted above, a medicine ejection device according to the invention is a medicine ejection device which ejects a medicine to be inhaled by a user via a suction port, comprising:

a flow path member linked to the suction port and capable of forming an air flow path in which the medicine is guided by the inhalation by the user to the suction port, and

an internal wall face constituting member which is intended for formation of the internal wall of the air flow path and allows detachable fitting in the flow path member.

Also in view of the problem noted above, another medicine ejection device according to the invention is a medicine ejection device which ejects a medicine to be inhaled by a user via an suction port, comprising:

a flow path member linked to the suction port and capable of forming an air flow path in which the medicine is guided by the inhalation by the user to the suction port,

a medicine ejection portion which ejects the medicine to the air flow path formed in the flow path member, and

a removing unit for removing the medicine delivered from the medicine ejection portion and having stuck to the internal wall of the air flow path.

In a medicine ejection device according to the invention, as it is provided with a detachable internal wall face constituting member which can form a new internal wall face within the air flow path, the stuck medicine can be easily removed if the user detaches the internal wall face constituting member after inhalation. As a result, the air flow path can be kept clean.

In another medicine ejection device according to the invention, as it permits removal of the medicine stuck within the air flow path after the use of the medicine ejection device, the air flow path within the device can be kept sanitary. Also, the trouble of washing or replacing a member forming the air flow path every time can be reduced.

Further features of the present invention will become apparent from the following description of exemplary embodiments with reference to the attached drawings.

### BRIEF DESCRIPTION OF THE DRAWING

FIG. 1 shows an external view of an inhaler in one embodiment of the present invention;

FIG. 2 is a block diagram illustrating the electrical configuration of the inhaler shown in FIG. 1;

FIG. 3 is a flow chart showing the operation of the inhaler;

FIG. 4 shows a perspective view of an inhaler in a second embodiment of the present invention;

FIG. 5 shows a section of the inhaler shown in FIG. 4;

FIG. 6 shows a modified version of the inhaler shown in FIG. 4, illustrating a section of the inhaler in which a film 12 is disposed to the area of a suction port 2;

FIG. 7 shows a perspective view of an inhaler in a third embodiment of the present invention;

FIG. 8 shows a section of the inhaler shown in FIG. 7;

FIG. 9 illustrates the inhaler shown in FIG. 7 as viewed from an air inlet 4 in a state in which a sheet 15 is folded back;

FIG. 10 shows a section of the vicinities of an air flow path in an inhaler in a fourth embodiment of the present invention;

FIG. 11 shows a modified version of the inhaler shown in FIG. 10;

FIG. 12 shows a perspective view of an inhaler in a fifth embodiment of the present invention;

FIG. 13 illustrates the inhaler shown in FIG. 12 as viewed from the suction port 2 side;

FIG. 14 shows an enlarged view of the save position for a wiper 25 shown in FIG. 13; and

FIG. 15 is a block diagram illustrating the electrical configuration of the inhaler shown in FIG. 12, including a stepping motor 27.

### BEST MODE FOR CARRYING OUT THE INVENTION

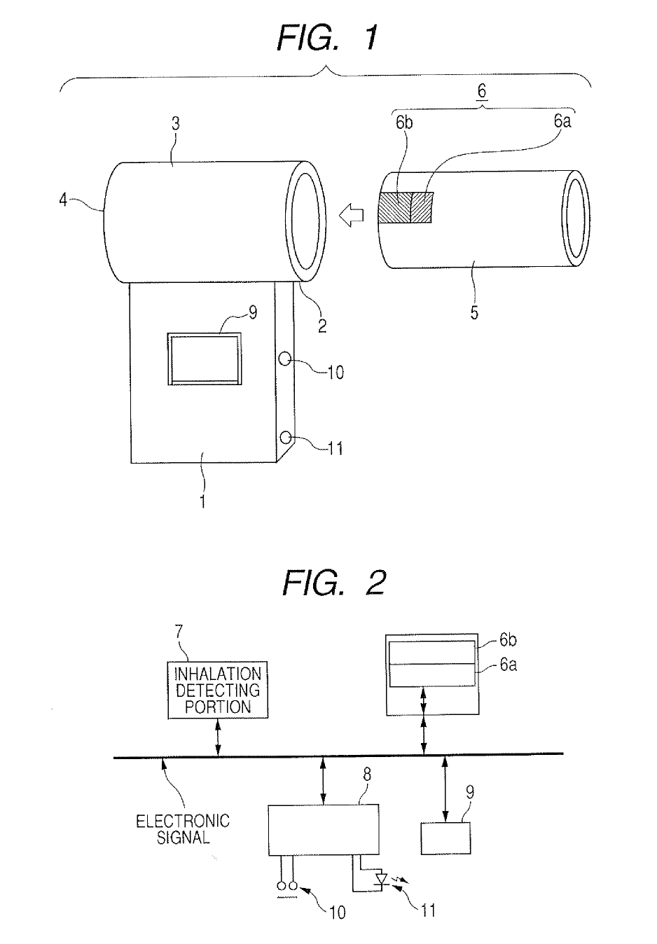

A mode for carrying out the invention embodied in a medicine ejection device will be described with reference to an inhaler, an example made portable for its user. FIG. 1 shows the configuration of the inhaler in one embodiment of the present invention. FIG. 2 is a block diagram illustrating the electrical configuration of the inhaler shown in FIG. 1. The inhaler has a configuration in which a flow path member 3 and a suction port (mouthpiece) 2, to be fitted to the mouth or nose of the user for inhalation, are fitted to a device body 1. At one end of the flow path member 3, an opening (air inlet 4) is provided to generate an air flow in the flow path member 3 when the user inhales from the suction port 2. The other end of the flow path member 3 is linked to the suction port 2. When the user inhales, an air flow is generated from the air inlet 4 toward the suction port 2. Then, ejecting a medicine into the air flow path in the flow path member 3 causes the medicine to be carried toward the suction port 2 and then into the user's mouth.

To be noted here, the “flow path member” in this specification means a member which constitutes the air flow path, which is the passage of the medicine from the point where the medicine is ejected within the medicine ejection device to the suction port. Thus, a space formed in the flow path member constitutes the “air flow path”.

However, part of the medicine ejected into the air flow path is feared to remain stuck to the internal wall of the air flow path. If the flow path member 3 and the suction port 2 are made detachable from the device body, the member can be washed every time. However, this inhaler has a mechanism which removes any medicine stuck to the internal wall of the air flow path, and accordingly the flow path member 3 need not be washed every time. The material to constitute the flow path member 3 is subject to no particular limitation, but can be selected as desired according to the specification of the medicine ejection device. Typical examples include plastic, metallic or rubber members. Incidentally, the flow path member and the suction port can be configured either as mutually linkable separate members or as an integrated body.

A controller 8 shown in FIG. 2, disposed within the device body 1, is a CPU which controls the operations of the whole inhaler including the driving of an ejection head 6a. When a signal of detection of inhalation is output from a pressure sensor 7 for detecting a negative pressure generated in the air flow path by the user's inhalation, the controller 8 transmits a drive signal to the ejection head 6a to start the delivery of the medicine in synchronism with the detection signal. In addition to these, a display unit 9 to enable the user to visually recognize any desired information on the operation of the inhaler, ejection conditions and the like may also be disposed. Reference numeral 10 denotes a power switch and 11, a power indicator LED which is lit when power supply to the inhaler is on.

According to the invention, the medicine ejection portion (ejection head) has an ejection pressure generating element of any desired type. Conceivable examples of the ejection pressure generating element included an electrothermal transducer which provides thermal energy to the medicine or an electromechanical transducer which provides mechanical energy to it. Typical examples of the medicine ejecting method include a method by which the medicine is given thermal energy by an electrothermal transducer and ejected from the ejection nozzle (thermal jet system) and another method by which the medicine is ejected from the ejection nozzle by using the vibratory pressure of an electromechanical transducer (e.g. piezoelectric element) which gives mechanical energy to the medicine. The ejecting method can be selected according to the type of the medicine and other factors.

When the thermal jet system is used, the bore of the ejection nozzle, the calorific value of thermal pulse used for ejection, and the size precision and reproducibility of a micro-heater as the electrothermal transducer can be increased for each individual liquid ejection unit. For this reason, it is possible to achieve a narrow distribution of drip diameters. The production cost of the head is low, and this enhances the applicability to small units which require frequent head replacement. Therefore, especially where the medicine ejection device is required to be portable and handy, a thermal jet ejection device can be used.

In this invention, the ejection head may be configured either as a medicine ejection cartridge integrated with a reservoir for accommodating the medicine or as a separate item from the reservoir. Although the medicine ejection cartridge is integrated with an internal wall face constituting member 5, to be described in more detail afterwards in the illustration in FIG. 1, usually a fitting portion which enables the cartridge to be fitted to the device body 1, and the ejection head is directed into the air flow path so that it can eject the medicine into the air flow path.

The concept of the medicine in the context of the invention is not limited to medicines which are pharmaceutical compounds manifesting pharmacological and physiological actions, but also covers, in addition to pharmaceutical compounds, contents intended as components for scenting and flavoring, dyes and pigments. The medicine may be either liquid or powder.

The medicine for use in this invention is a liquid medicine or a liquid medium containing a medicine. The liquid medicine may contain any desired additive. The state of the medicine in the liquid may be dissolved, dispersed, emulsified, suspended or slurried, more preferably uniformized in the liquid.

When a medicine liquid is to be used as the medicine, it is preferable for the main medium of the liquid to be water or an organic substance, and water as the main medium is more preferable in view of the circumstance where it is intended for administration to a living body.

The mechanism for removing the medicine stuck within the air flow path, which is a characteristic feature of the invention, may be one or another of the following propositions. One is the formation of a new internal wall face over the internal wall face of the air flow path to which a medicine remains stuck after the medicine is ejected. Another is to dispose a wiping unit to clean the internal wall face of the air flow path to which a medicine remains stuck after the medicine is ejected.

The internal wall face of the air flow path should be sanitary because it comes into contact with the air flow which carries the medicine, and these arrangements enable the medicine to be ejected into the air flow path which will remain clean next time it is used. Moreover, since the flow path member 3 need not be replaced or detached and cleaned every time, they are user-friendly.

Specific embodiments will be described below.

### Embodiment 1

As shown in FIG. 1, the inhaler in this embodiment has the device body 1, the flow path member 3 and the mouthpiece 2 supported by the device body 1, and the internal wall face constituting member 5 detachably fitted into the flow path member 3.

The internal wall face constituting member 5 is formed to be detachable from the flow path member 3 and the mouthpiece 2, and the mouthpiece 2 is formed to be detachable from the device body 1. As at least a part of the internal wall face constituting member 5 is made of a flexible material, this member can be easily attached to or detached from the flow path member 3 and the mouthpiece 2.

To be noted here, the “internal wall face constituting member” in this specification can be anything that has an action capable of preventing the medicine from directly sticking to the internal wall of the flow path member and, in particular where it is configured to be detachable from the flow path member, it means a member constituting the internal wall of the air flow path when it is fitted to the flow path member. Thus, as is evident from FIG. 1, while part of the surface of the flow path member 3 constitutes “the internal wall of the air flow path” when the internal wall face constituting member 5 is not fitted, when the internal wall face constituting member 5 is fitted, this member constitutes the internal wall of the air flow path. Incidentally, though it is preferable for the internal wall face constituting member to span the whole internal wall face of the flow path member, it need not fully cover the whole face depending on the extent of the contamination, but may cover only a part.

Using such an inhaler, the user may inhale the medicine three times a day for instance. And the internal wall face constituting member 5 is replaced on every occasion of inhalation. Further, the flow path member 3 and the mouthpiece 2 are supposed to be replaced for use once a day for instance.

It is preferable for the material of the internal wall face constituting member 5 to be, though not limited to, what can be easily attached to and detached from the flow path member 3 and does not chemically react with the medicine even if the medicine sticks to it. Typically, it can conceivably be a plastic material or a rubber material. A hollow cylindrical member like the illustrated one would be easy to fabricate by resin molding or otherwise.

Replacing the internal wall face constituting member 5 on every occasion of inhalation would enable the medicine to be ejected to a clean air flow path every time.

It is preferable for the ejection head 6a and the reservoir 6b constituting the medicine ejection portion to be configured integrally with the internal wall face constituting member 5 as shown in FIG. 1. The reason is that, where the ejection head and the reservoir are discarded every time, the ejection head and other replaceable elements can be removed in a single procedure at the same time as the removal of the internal wall face constituting member 5.

The shape of the air flow path to be formed inside the flow path member 3 or the internal wall face constituting member 5, though shown to be cylindrical in FIG. 1, is not limited to this, but may be prismatic or otherwise.

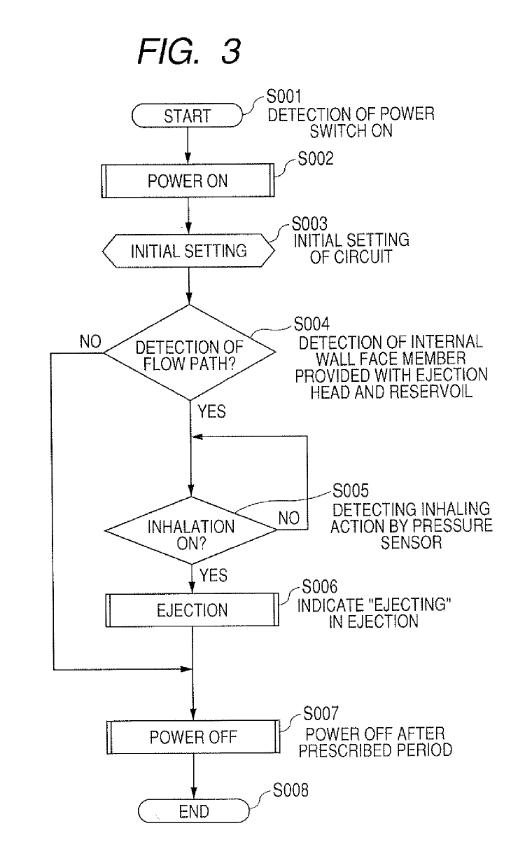

FIG. 3 is a flow chart showing the operation of the inhaler.

First, the user joins the flow path member 3 and the mouthpiece 2 with the device body 1. Further, the user joins the ejection head 6a and the internal wall face constituting member 5 having the reservoir 6b built into it with the mouthpiece 2.

The power switch is pressed by the user (step S001), and a power ON state is achieved (step S002). The turning-on of power supply causes the power indicator LED 11 to be lit. The controller 8 executes initial setting of the internal functions of the device (step S003). After that, it is detected whether or not an internal wall face constituting member 5 is fitted according to an electric signal (step S004). If no internal wall face constituting member 5 can be detected, it is determined to mean NO, and the processing shifts to step S007 to automatically turn off power supply and ends at step S008.

If any internal wall face constituting member 5 is detected, it is determined to mean YES, and the processing shifts to step S005. At step S005, the patient's inhaling action is detected with the pressure sensor 7. If no inhaling action by the patient can be detected, it is determined to mean NO, and the processing returns to step S005. If an inhaling action by the patient can be detected, it is determined to mean YES, and the processing shifts to step S006. At step S006, “Ejecting” is indicated on the display unit 9, and control is so effected as to eject a prescribed dose of the medicine accommodated in the reservoir 6b from the ejection head 6a, followed by a shift to step S007. At step S007 the controller 8 turns off power supply to the device, and the processing ends at step S008. As power supply to the device is turned off, the power indicator LED goes off, and every indication on the LCD display is also turned off. After the end of inhalation, the user detaches the ejection head 6a and the internal wall face constituting member 5 having the reservoir 6b built into it from the mouthpiece 2 and the device body 1. Also, after the three rounds of inhalation a day for instance, the user removes the mouthpiece 2 from the device body 1 to end the operation.

### Embodiment 2

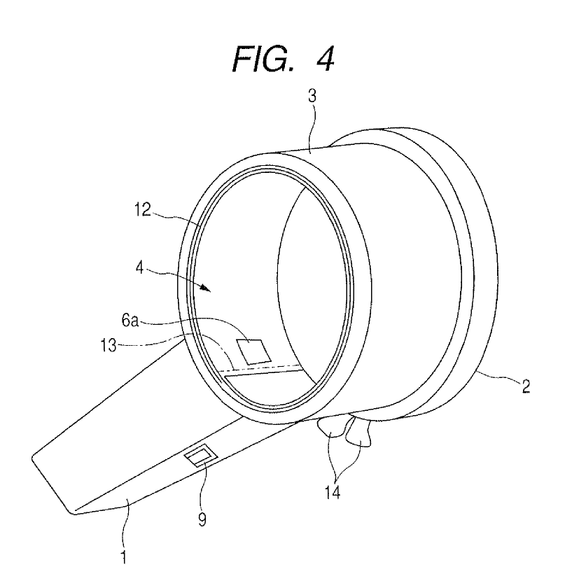

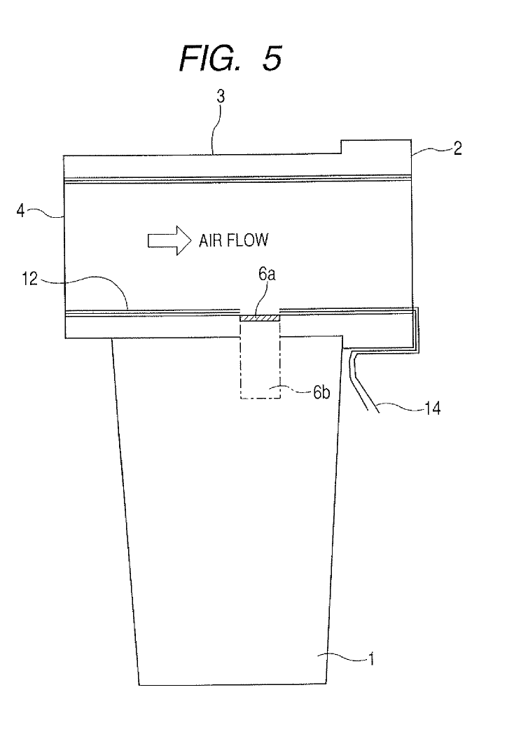

A perspective view of an inhaler in a second embodiment of the present invention is shown in FIG. 4. FIG. 5 shows a section of the inhaler shown in FIG. 4.

In this embodiment, the internal wall face constituting member 5 referred to in Embodiment 1 is replaced with a thin film 12. The film 12 serving as the internal wall face constituting member is in tight contact with the internal wall face of the air flow path which constitutes part of the surface of the flow path member 3, and therefore scarcely affects the shape of the air flow path. Accordingly, no matter whether the air flow path is provided with the film 12 or not, the air flow generated by inhalation does not vary, but a similar state of inhalation can be realized. After the inhalation ends, when the user peels off the film 12 to which the medicine has stuck, the stuck medicine can be removed from the air flow path, and a new internal wall face of the air flow path is formed after the film is peeled off.

It is more preferable here for a plurality of layers of the film 12 to be stacked in advance. By peeling off one layer of film after each occasion of inhalation, the frequency of replacing the flow path member 3 and the mouthpiece 2 can be reduced.

As shown in FIG. 4, the inhaler in this embodiment has the inhaler body 1, the flow path member 3 supported by the device body 1 and the film 12 in tight contact with the internal wall face of the flow path member 3 to serve as the internal wall face constituting member. The ejection head 6a and the reservoir 6b (together constituting a medicine ejection cartridge 6) is fitted to the device body 1, and the ejection head 6a is so oriented as to be able to eject the medicine into the air flow path.

An air flow is generated within the flow path member 3 by the user's inhalation.

The film 12 is rolled as shown in FIG. 4, and is in tight contact with the internal wall face of the air flow path which constitutes part of the surface of the flow path member 3 in a plurality of layers (two layers are shown in FIG. 4). So that the user can peel off one layer of the film at a time, each layer of the film 12 is provided with a film pinch 14. The user peels off an equivalent length of the film to one round of the internal wall face of the air flow path by pulling the film pinch 14 after the end of inhalation. In this way, it is possible to eject the medicine to a clean air flow path on the next occasion of inhalation. It is advisable to make a cut 13 to facilitate peeling off one layer at a time.

And when the final part of the film has been smeared by inhalation, the flow path member 3 is replaced. It is sufficient to replace the medicine ejection cartridge 6 with the ejection head 6a and the reservoir 6b built into it once a week if a quantity of the medicine for a week's consumption is stored in the reservoir 6b. For instance, if inhalation is to be done three times a day, stocking 21 layers of the film would enable the flow path member 3 and the medicine ejection cartridge 6 to be replaced at the same time.

Description of the electrical configuration of the inhaler and the operating sequence of the device will be dispensed with as they are the same as in Embodiment 1.

There is not particular limitation of the threading of the film. It may be continuous to constitute a roll via the cut 13 as shown in FIG. 4, or the layers of film may be separated from one another. The position of the film pinch 14 also can be freely selected. Although it is disposed on the suction port 2 side in the arrangement illustrated in FIG. 4, it can as well be provided on the air inlet 4 side. This is a more preferable arrangement because the film pinch 14 would not obstruct inhalation.

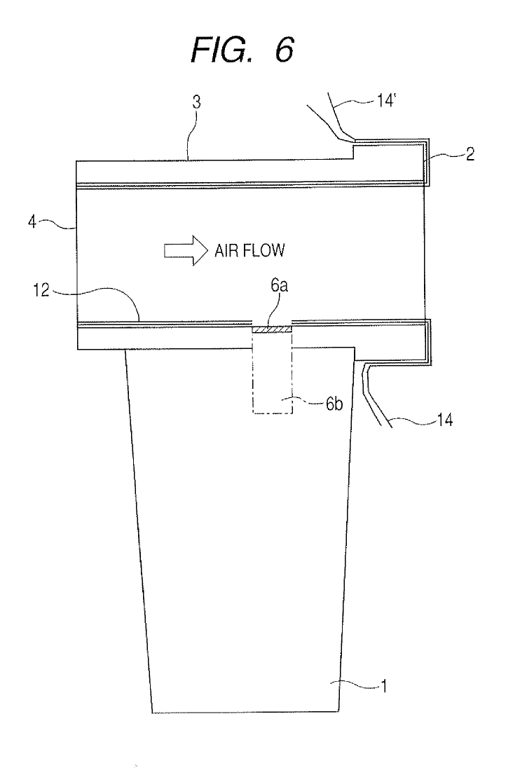

On the other hand, it is also possible to extend the film 12 in tight contact with the internal wall face of the air flow path to the area of the suction port 2. A section of the inhaler in such an embodiment is shown in FIG. 6. As the film 12 is present even in the part where the user's mouth comes into touch and the film is peeled off after use, the suction port can be kept clean. In this case, with a view to greater ease of peeling off the film, a plurality of film pinches may be provided for each layer (film pinches 14 and 14′).

### Embodiment 3

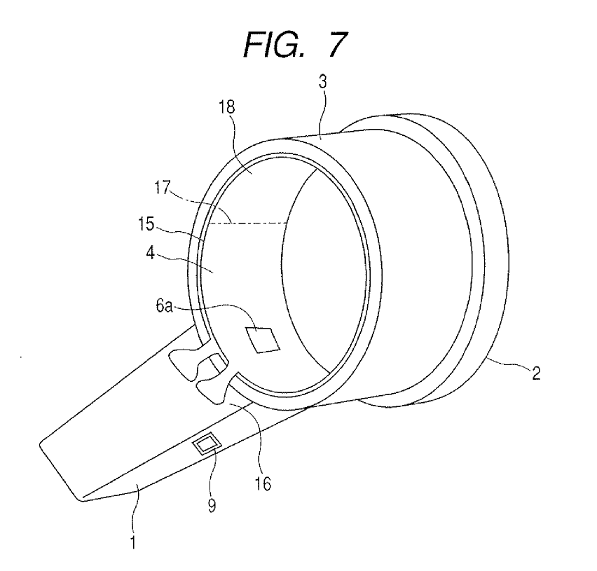

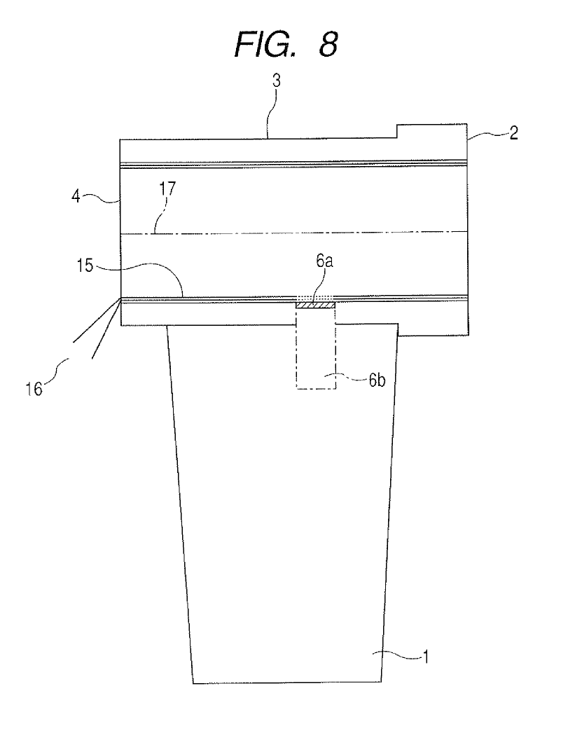

A perspective view of an inhaler in a third embodiment of the present invention is shown in FIG. 7. FIG. 8 shows a section of the inhaler shown in FIG. 7.

The configuration in this embodiment does not involve peeling-off of the film 12 as in Embodiment 2, but is one folding back a sheet 15 serving as the internal wall face constituting member within the air flow path after inhalation. A plastic sheet having a degree of softness permitting easy manual folding can be used as the sheet 15. The sheet 15 is in tight contact with the internal wall face of the air flow path and hardly affects the shape of the air flow path. Therefore, no matter whether the air flow path is provided with the sheet 15 or not, the air flow generated by inhalation does not vary, but a similar state of inhalation can be realized. After the inhalation ends, when the user lifts a sheet pinch 16, the sheet 15 is folded along a cut 17, and the faces which constituted the internal wall face of the air flow path during inflation come into contact. As a result, the stuck medicine is removed from the internal wall surface of the air flow path, and a new internal wall face of the air flow path is formed. The air flow which carries the medicine on the next occasion of inhalation will arise on the clean internal wall face free from the sticking of the medicine.

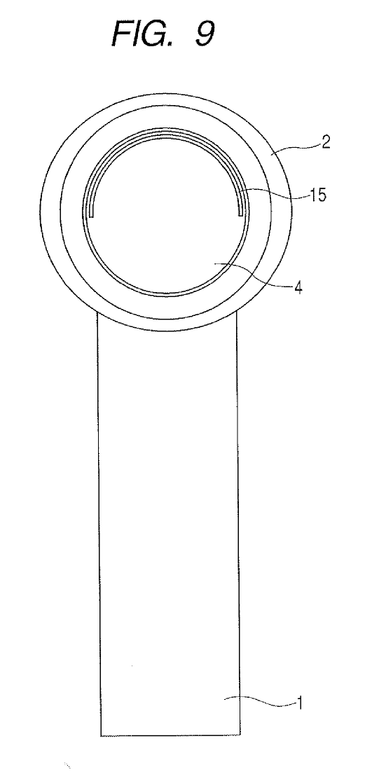

FIG. 9 illustrates the inhaler as viewed from the air inlet 4 in a state in which the sheet 15 is folded back. Cuts are made in two opposite positions within the air flow path to enable the sheet 15 to be folded in the reverse direction in a stroke if lifted with a force not weaker than a prescribed level.

Though not shown in the figure, if not only the sheet pinch 16 on the air inlet 4 side but also another is disposed on the suction port 2 side and both pinches are lifted, the sheet 15 can be folded more easily.

An adhesive 18 may be applied in advance onto the upper half of the air flow path internal wall above the cut 17. In this case, sheets 15 are adhered to one another to prevent the trouble of the sheet 15 being unfolded by a wrong action after inhalation without being noticed. The material of the adhesive 18, though not particularly required to be so, may preferably be not so viscous as to let it stick to a finger which might come into contact. Something like the glue on the back side of a postage stamp, made of polyvinyl alcohol, could advisably be applied in advance. This material, which is made adhesive by contact with liquid, is not adhesive before inhalation, and is convenient because the sticking of the ejected liquid medicine to the sheet 15 gives it an adhesive force.

A plurality of layers of the sheets 15 may be provided and folded back repeatedly. In that case, each sheet needs to be provided with a pinch.

### Embodiment 4

Embodiment 2 and Embodiment 3 use a method by which a pinch is utilized for peeling off or folding back the film or the like, but a configuration which allows the user to automatically peel off the film by a simple action can be adopted as well.

A method of winding up the film in a roll shape will be described as an example of such inhaler.

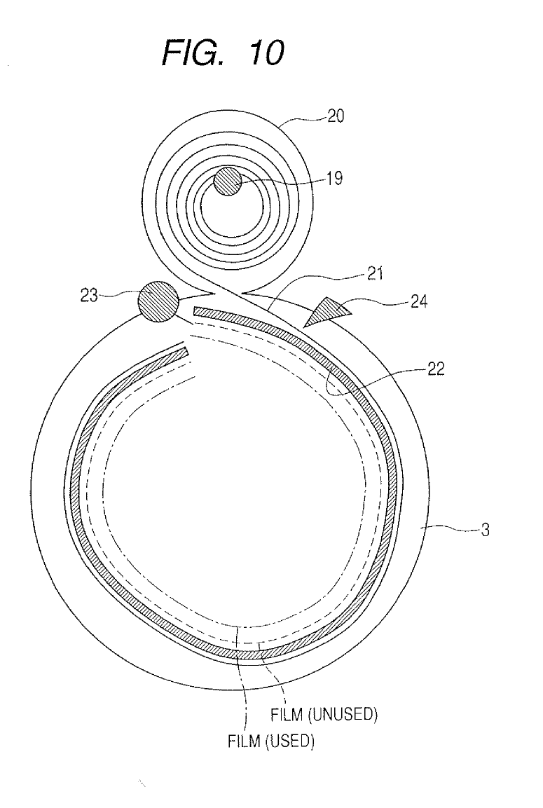

FIG. 10 shows a part of the section of the inhaler as viewed from the air inlet 4 with the device body 1 being excluded. A take-up roll 19 and a roll chamber 20 are arranged outside and above the flow path member 3 and connected to the flow path member 3. The used film is wound up into the roll 19 from inside the air flow path. The transfer of the film from inside the air flow path into the roll chamber 20 is carried out in the following manner. Within the air flow path, a support plate 22 turns to urge the film to transfer. The roll 19 has a leader film 21, and the leader film 21 is kept in contact with the support plate 22 in advance; the rotation of the support plate 22 moves the used film to be spliced with the leader film. By pressing a stud 23, a claw provided on the stud 23 is caused to release the used film from its stacked state, and the used film is spliced by the stud with the leader film on the support plate. Advisably, a splicing agent may be provided near an end of the film to facilitate splicing of the used film then with the leader film. The splicing agent may be any adhesive, or splicing may as well be accomplished by an electrostatic action. The passage for the stud 23 is provided in advance in the external circumferential face of the flow path member 3. This enables rotation of the roll 19 to rotate the interlocked support plate 22 and the further interlocked used film to transfer to be wound up by the roll 19. The arrangement is such that the stud 23 is then moved away from the film by a block 24 and returns to its initial position as it rotates. When a series of repeated actions take place, the previous used film serves as the leader film for the next round.

The user himself or herself may control the roll 19, or a rotary mechanism such as a motor may be provided to automatically control it via a controller in accordance with a program. The roll 19 is provided with a lever for controlling the rotation when the user is to operate it, and the lever is turned to rotate the roll 19, resulting in the execution of the operation described above.

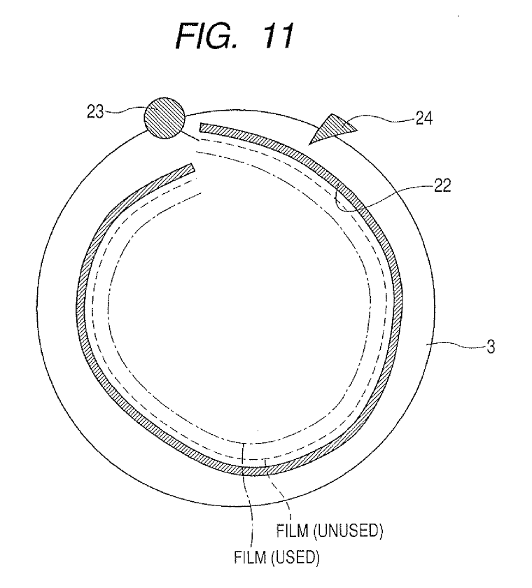

A system of winding up within the flow path member 3 is shown in FIG. 11 as an example of winding-up system. As in the case of FIG. 10, the used film is released by the stud 23 from the stacked film, and the used film is transferred by the stud 23 along the external circumference of the support plate 22. The passage of the stud 23 and the block 24 similarly configured would enable the stud 23 to return to its initial position.

This enables the used film to be transferred to the external circumferential face of the support plate 22, and the series of repeated actions cause the used films to be stacked on the external circumferential face of the support plate 22. This operation, too, may be automatically accomplished by a rotary mechanism such as a motor or a gear rail via a controller in accordance with a program.

The system of winding up the film within the flow path member 3 may as well have a double track structure in which a rail or lead member for guiding the stud 23 is provided over one and half turns around the external circumferential face of the flow path member 3 and a half turn may be changed over in a switchback way. This enables the used film to transfer one full turn to be replaced by a new face by only a half turn of the stud 23 traveling along the external circumferential face of the flow path member 3.

### Embodiment 5

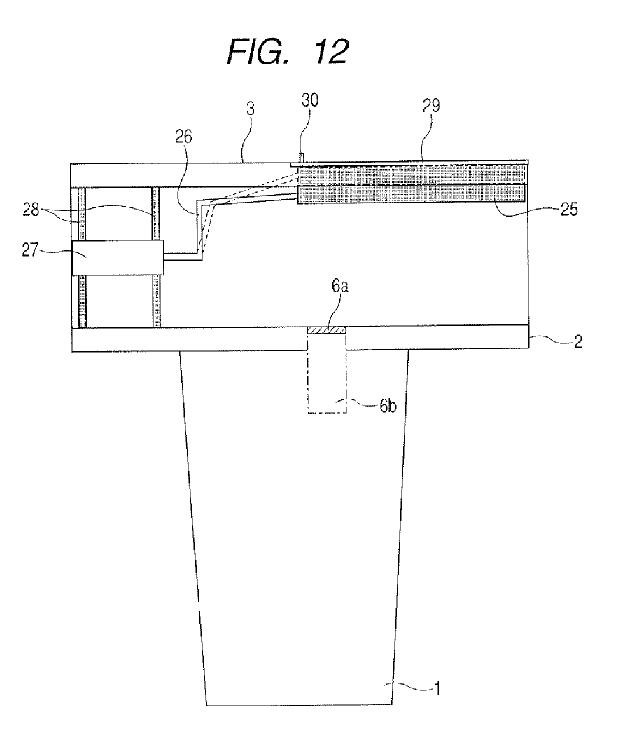

A section of an inhaler in a fifth embodiment of the present invention is shown in FIG. 12. The inhaler in this embodiment has a wiper 25, in contact with the internal wall face of the flow path, as a unit for removing the medicine stuck to the internal wall face of the air flow path formed by the flow path member 3. The medicine stuck to the internal wall face of the air flow path is wiped off with the wiper 25. There is no particular limitation to the configuration of the wiper 25, which needs only the capability of wiping off the medicine. The wiper 25 may have an absorber capable of sucking the medicine. Or it may have a blade-shaped structure provided with a separate medicine collecting mechanism.

Incidentally, a suction pipe having a suction pump may be used in place of the wiper 25.

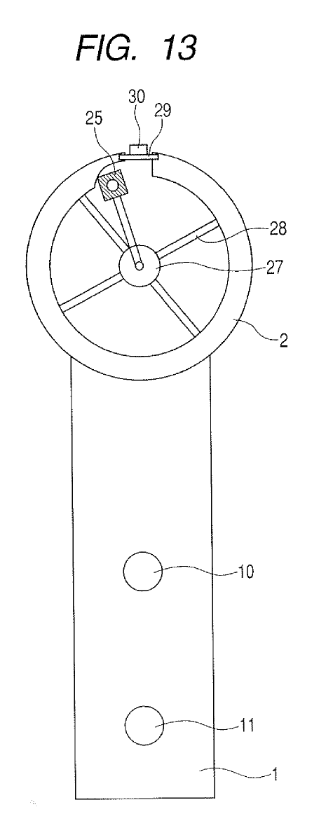

The wiper 25 is supported by a wiper supporting rod 26. The wiper supporting rod 26 has a spring mechanism, which is the angled part in FIG. 12, and is so urged that the wiper 25 comes in constant contact with the internal wall face of the air flow path. Incidentally, in FIG. 12, the state in which the wiper 25 and the wiper supporting rod 26 are represented by solid lines is a state of being in contact with the internal wall face of the flow path, and the state in which they are represented by broken lines is a state of being accommodated in the save position to be described afterwards with reference to FIG. 13. When the user has finished inhalation and the ejection of the medicine is stopped, the controller 8 transmits an operation signal to a stepping motor 27 supported by motor supporting pillars 28. The wiper 25 is caused by the stepping motor 27 to rotate along the internal wall face of the air flow path in a full turn to wipe the stuck medicine. (FIG. 13 shows a block diagram of the electrical configuration of the inhaler in this embodiment, including the stepping motor 27.)

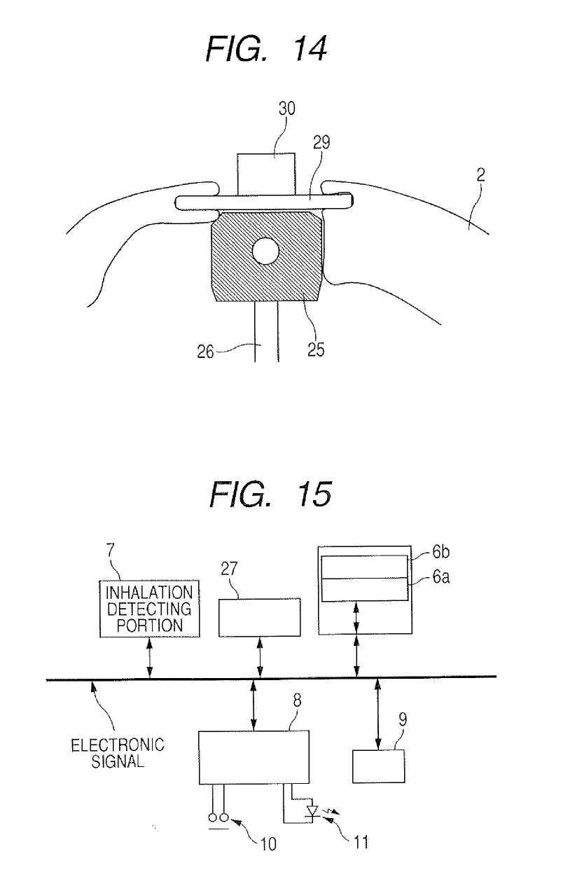

FIG. 13 illustrates the inhaler shown in FIG. 12 as viewed from the suction port 2 side. Part of the flow path member 3 and the suction port 2 is a partially chipped structure to accommodate the wiper 25, and a slide cover 29 is positioned there. While the user is inhaling, the wiper 25 is accommodated in a space near the slide cover 29 (FIG. 14).

The controller 8 so controls the revolution of the stepping motor 27 as to cause the wiper 25 to make a full turn counterclockwise in FIG. 13, while wiping the internal wall face of the air flow path, from the position of the wiper 25 shown in FIG. 14 (the save position). This results in so controlling the wiper 25 as to wipe off the medicine stuck to the internal wall face of the flow path and return to the save position.

The repetition of the rotating operation of the wiper 25 every time the delivery ends enables the internal wall face of the air flow path to be kept in a clean state.

And, after three rounds of inhalation a day for instance, the user holds a slide knob 30 and slides the slide cover 29 toward the suction port 2. The user wipes the wiper 25 with a towel or the like through an opening bored in the flow path member, and further cleans it by washing with alcohol or the like. After that, the user returns the slide cover 29 to its original position. This enables the wiper 25 to be returned to a clean state.

As the wiper 25 is intended for wiping off the stuck medicine, it is adequate for the wiper to be so arranged as to be able to wipe the area farther toward the suction port 2 than the ejection head 6a. However, if it is made long enough to be able to wipe the ejection head 6a as well as shown in FIG. 12, it will be more favorable because wiping of the ejection nozzle face of the ejection head can be accomplished at the same time as the wiping of the internal wall face of the air flow path. Thus, it is preferable for the wiper 25 to be as long as the distance from the position of the ejection head 6a to the suction port 2 within the flow path member 3.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2006-196327, filed Jul. 19, 2006 and No. 2007-150303, filed Jun. 6, 2007, which are hereby incorporated by reference herein in their entirety.

1. A medicine ejection device which ejects a medicine to be inhaled by a user via a suction port, comprising:
a flow path member linked to the suction port and capable of forming an air flow path in which the medicine is guided by inhalation by the user to the suction port; and
an internal wall face constituting member which serves to form an internal wall of the air flow path and allows detachable fitting in the flow path member.

2. The medicine ejection device according to claim 1, further comprising:
a medicine ejection portion which ejects the medicine to the air flow path formed in the flow path member.

3. The medicine ejection device according to claim 2, wherein the medicine ejection portion is formed integrally with the internal wall face constituting member.

4. The medicine ejection device according to claim 1, wherein the internal wall face constituting member is flexible.

5. The medicine ejection device according to claim 1, wherein the internal wall face constituting member has a film positioned to come into tight contact with the inside of the flow path member.

6. The medicine ejection device according to claim 5, wherein the film in a state of being stacked in a plurality of layers comes into tight contact with the inside of the flow path member.

7. The medicine ejection device according to claim 1, wherein the internal wall face constituting member has a sheet which can be folded back within the air flow path.

8. A medicine ejection device which ejects a medicine to be inhaled by a user via a suction port, comprising:
a flow path member linked to the suction port and capable of forming an air flow path in which the medicine is guided by inhalation by the user to the suction port;
a medicine ejection portion which ejects the medicine to the air flow path formed in the flow path member; and
a removing unit for removing the medicine ejected from the medicine ejection portion and stuck to an internal wall of the air flow path.

9. The medicine ejection device according to claim 8, wherein the removing unit is a wiping unit which cleans the internal wall of the air flow path after the ejection of the medicine.

10. The medicine ejection device according to claim 9, wherein the wiping unit also wipes an ejection nozzle face of the medicine ejection portion at the same time as cleaning the internal wall of the air flow path.

11. The medicine ejection device according to claim 2, wherein the medicine ejection portion has an electrothermal transducer which provides thermal energy to the medicine or an electromechanical transducer which provides mechanical energy to the medicine.
